(19) United States
(12) Patent Application Publication
(10) Pub. No.: US 2026/0206988 A1
(43) Pub. Date: Jul. 23, 2026

(54) Smart Pillow System

(71) Applicant: Dura Incorporation, Sunnyvale, CA (US)

(72) Inventor: Zhiqiang Yang, Sunnyvale, CA (US)

(21) Appl. No.: 19/142,197

(22) PCT Filed: Dec. 29, 2023
(86) PCT No.: PCT/US2023/086559

§ 371 Date: Jun. 21, 2025

Related U.S. Application Data
(60) Provisional application No. 63/436,569, filed on Dec. 31, 2022.

(51) Int. Cl. A47G 9/10

(52) U.S. Cl. CPC A47G 9/1027; A47G 9/109

(57) ABSTRACT

A smart pillow system deformable by a controller for maintaining the cervical spine of a user who is a human being in a neutral alignment despite changes in the position of the user from lying on his or her side or on his or her back, comprising: at least one inflatable bladder, a controller, the controller comprising an air pump to increase air pressure in the at least one inflatable bladder, a pressure gauge to measure air pressure in the at least one inflatable bladder, and a valve to release air from the at least one inflatable bladder, and a microprocessor operatively connected to the air pump, pressure gauge, and valve to operate the same, wherein the controller is adapted to measure the pressure of air in the first inflatable bladder and increases or decreases the pressure of the air in the at least one inflatable bladder.

### CROSS-REFERENCE TO RELATED APPLICATIONS

This Patent Cooperation Treaty application claims priority from U.S. provisional application 63/436,569 filed by applicant Dura Incorporation on 31 Dec. 2022. The disclosure of U.S. provisional application 63/436,569 is incorporated by reference for all purposes allowed by law.

### TECHNICAL FIELD

The field of the disclosure is that of that of devices for aiding sleep and relates generally to an adjustable pillow that supports the head of a person as he or she sleeps.

### BACKGROUND OF THE INVENTION

A wide variety of different sleep systems are currently available. Such sleep systems may comprise all aspects of a bedding assembly including, but not limited to, mattresses, box springs, foundation units, bed frames, pillows, mattress pads, linens and, more generally, to any type of sleep product that influences a person's sleep. The characteristics of a suitable sleep system for a person depend on a number of factors, including, but not limited to, the physical attributes of the person (e.g., weight, height, body dimensions, weight distribution, etc.), preferred sleeping positions (e.g., sleeping on back, side, front, etc.), sleeping habits and so on.

Two very different primary components of sleep systems affect a person's overall sleep experience: support and comfort. A sleep system delivers support to a person by holding the person in a proper postural alignment, while increasing comfort by evenly redistributing the person's body weight across a wide area so as to relieve undue pressure on a part of the body.

The proper postural alignment of a person is one in which the person's spine maintains its natural anterior and posterior curvature while remaining straight or in a plane in the lateral and medial sense. Unnatural curvature, whether in the anterior/posterior sense, or in the lateral and medial sense, is likely to lead to discomfort and unsatisfactory sleep.

A person generally tends to change position when asleep or trying to sleep. In particular, a person may switch between prone and supine positions and between sleeping on the right side of the body and the left side of the body. A standard pillow will not tend to provide the support required for natural spinal curvature in the various positions. The person, if awake, may adjust one or more pillows as needed to achieve better support but this tends to require a conscious effort that is inconsistent with sleep. The person may change position while asleep and render the pillows ineffective for achieving the proper support of natural spinal curvature.

Thus, a pillow system that is most suitable for a person is a pillow system that provides a combination of comfort and support to the person. Further, people vary considerably in their physical attributes and sleeping habits. A pillow system that can accommodate itself to these differences would provide better comfort and support. The pillow system, furthermore, should be durable and affordable.

### SUMMARY OF THE INVENTION

The disclosure provides a smart pillow system that senses the orientation of a sleeping person's body and adjusts the shape of a pillow according to that orientation. The shape of the pillow is adjusted in order to support the person's head in a way that is consistent with natural curvature of the spine.

An embodiment of the smart pillow system is deformed by a control system or controller in order to maintain the cervical spine of a user, who is a human being, in a neutral alignment despite changes in the position of the user from lying on his or her side or on his or her back. This embodiment comprises at least one bladder that is capable of holding air. An encasement layer may be configured to enclose the at least one bladder to cushion the at least one bladder and provide a comfortable surface for contacting the user's head. The embodiment further comprises a control system or controller, a pump to increase pressure of the air in the bladder, a pressure gauge to measure pressure of the air in the bladder, and a valve to release air from the bladder. The controller comprises a microprocessor operatively connected to the air pump, pressure gauge, and valve to operate the same and expand or deflate the bladder as needed to raise or lower the head of a user resting her head on the smart pillow system.

The fluid preferably is air because air is available from the exterior of the smart pillow system for pumping into the bladder and air may be released from the bladder to its surroundings without any difficulty. Use of another fluid, such as water, would require a reservoir for providing and receiving the fluid.

The controller is adapted to measure the pressure of the air in the at least one bladder and, based on the detected positioning of the user's body, increases or decreases the pressure of the air in the at least one bladder in order to increase or decrease a height of the at least one bladder as needed to maintain the neutral alignment of the cervical spine of the user.

In preferred configurations, the smart pillow system may have two or more bladders. The bladders may be spaced apart horizontally in order to gently rock the head of the user by selective inflation or deflation of the bladders when the controller detects snoring. For that reason, even numbers of bladders would be provided in order for the same number of bladders to be provided on either side of the smart pillow system.

Preferably, the bladders of the smart pillow system will form a depression for receiving the user's head. The bladders would be shaped with backs and arms laid out around a common void or cavity to form the depression for receiving the user's head. The smart pillow system may further comprise a second inflatable bladder, wherein the first inflatable bladder and the second inflatable bladder together define the hollow area between them.

In embodiments of the smart pillow system using what may be called the “delta pressure method” to detect the position of the user, the controller is configured to provide a measurement relating to a first pressure of the air in the at least one inflatable bladder when a user is lying with his or her head on the pillow with the at least one inflatable bladder underneath the head of the person and the person is in either a supine or a side position and a measurement relating to a second pressure of the air in the at least one inflatable bladder when the user is lying with his or her head on the pillow with the at least one inflatable bladder underneath the head of the person and the person is in the side or the supine position.

Thus, in the smart pillow system of the embodiments using the “delta pressure method,” the controller is configured to inflate the at least one inflatable bladder to a reference pressure prior to the user lying with his or her head on the pillow and prior to the controller providing the measurements of the first pressure of air and the second pressure of the air. The controller is configured to compare the measurements relating to the first and the second pressures and to assign the larger of the first and second pressures to the supine position and the smaller of the first and second pressures to the side position.

The controller may be configured to employ a table of pressures to be applied to the at least one bladder for each of the side and supine positions of the user wherein those pressures are determined by observation of the user in the respective positions in order to achieve neutral alignment of the cervical spine of the user. This will be called a “personalized method” for determining the pressures for the at least one bladder. A technician or other trained person may make the observations during an initialization stage.

The controller may also be configured to automatically calculate the pressure of the air in the at least one inflatable bladder to achieve the heights of the first inflatable bladder for the supine position and for the side position that will place the cervical spine of a human being in a neutral alignment for the supine position and for the side position. This will be called a “generalized method” of determining the pressures. In an embodiment of a generalized method called the “pressure averaging method,” the smart pillow system's controller is configured to calculate the pressure of the air in the at least one inflatable bladder to achieve the heights of the at least one inflatable bladder for the supine position and for the side position that will place the cervical spine of the user in a neutral alignment for the supine position and for the side position by averaging the measured pressures of air associated with the respective positions with the reference pressure. Other methods of determining the pressures appropriate to achieve the height of the at least one inflatable bladder for the side and supine positions include the “survey method” and the “shoulder width and weight/height method.”

The smart pillow system using the pressure system may further comprise a second inflatable bladder, wherein the at least one or first inflatable bladder and the second inflatable bladder together define the hollow area between them and the controller is configured to separately inflate or deflate the first and second inflatable bladders. A web may join the first and second inflatable bladders. The first and second inflatable bladders may be disposed, respectively, on right and left sides of the smart pillow system.

The smart pillow system may have the controller provided with a microphone and configured to detect the sound of snoring by the person and to inflate the first and second inflatable bladders unequally in order to rock the head of the person whereby the person will stop snoring.

The smart pillow system may further comprise a third inflatable bladder positioned beneath the first inflatable bladder and a fourth inflatable bladder beneath the second inflatable bladder wherein the controller is configured to inflate or deflate the third and fourth inflatable bladders in order to place the cervical spine of a human being in a neutral alignment for the supine position and for the side position.

In another preferred configuration of the smart pillow system according to the disclosure (using what may be called the “shoulder pressure method,” shoulder pressure sensors in or attached to the smart pillow system according to that configuration detect the pressures induced by the user's shoulders whereby the controller may determine the position of the user's body (supine or side position) and increase or decrease the pressure of the air in an at least one bladder in order to increase or decrease a height of the at least one bladder as needed to maintain the neutral alignment of the cervical spine of the user.

The embodiment of the smart pillow system using the “shoulder pressure method” to detect the position of the user may employ a table of pressures determined through observation for inflation of at least one bladder to the height appropriate to the position of the user in order to maintain the neutral alignment of the cervical spine of the user. Alternatively, the “pressure averaging method” or a “shoulder width and weight/height method” may be employed to provide the pressures to be applied for the supine and the side positions.

### BRIEF DESCRIPTION OF THE DRAWINGS

Other objects, features, and advantages of the present invention will become more fully apparent from the following detailed description of preferred embodiments, the appended claims, and the accompanying drawings in which:

FIG. 1 is an illustration of a person lying on her side with her head supported by a pillow and with her neck in neutral alignment. (When this disclosure refers to a user or person of one sex it is meant, as a way to avoid repetitious use of “his or her” and like formulations, to include users or persons of the opposite sex).

FIG. 2 is an illustration of the person lying on her back with her head supported by a pillow with her neck having its normal cervical curvature.

FIG. 3 is an illustration of the person lying on her back with her head supported by a pillow that is too high so that the person's neck no longer has its normal curvature.

FIG. 4 is an illustration of a person lying on her side with her head supported by a pillow that is too low so that the person's neck is no longer in neutral alignment.

FIG. 5 is a perspective view of a preferred embodiment of the smart pillow system according to the disclosure.

FIG. 6 is a partially exploded view of the preferred embodiment of the smart pillow system of FIG. 5.

FIG. 7 is a partially exploded view of the preferred embodiment of the smart pillow system of FIG. 5.

FIG. 8 is the partially exploded view of the preferred embodiment of the smart pillow system of FIG. 5 as shown in FIG. 7 but rotated around the vertical axis by about 120 degrees.

FIG. 9 is a perspective view of another preferred embodiment of the smart pillow system according to the disclosure showing some of its internal components. This preferred embodiment is similar to the smart pillow system of FIG. 5 but provides four bladders or air bags.

FIG. 10 is a perspective view of the preferred embodiment of the smart pillow system of FIG. 9, but rotated about the vertical axis to show the opposite side.

FIG. 11 is perspective view of the bottom of the preferred embodiment of the smart pillow system of FIG. 9, with the bottom tray omitted in order to view some of the inner components.

FIG. 12 is a perspective view of the top of the preferred embodiment of the smart pillow system of FIG. 9, with the top pillow exterior omitted in order to show some of the internal components.

FIG. 13 is a perspective view of the bottom of the preferred embodiment of the smart pillow system of FIG. 9, with the bottom tray removed to show some of the internal components.

FIG. 14 is a perspective view of the bladders or air bags of the preferred embodiment of the smart pillow system of FIG. 9.

FIG. 15 is a side view of the bladders or air bags of the preferred embodiment of the smart pillow system of FIG. 9.

FIG. 16 is a schematic diagram of the pressure sensor and valve assembly of the embodiment of a smart pillow according to FIG. 9.

FIG. 17 is a schematic of the pressure sensor and valve assembly of the embodiment of a smart pillow according to FIG. 9 but modified to provide for independent inflation or deflation of the top two bladders or air bags.

FIG. 18 is a schematic showing the components and their interrelationship of the preferred embodiments of the smart pillow system of either FIG. 5 or FIG. 9.

FIG. 19 is a flow chart showing how the control unit of the preferred embodiments of the smart pillow system of either FIG. 5 or FIG. 9 initializes with respect to the user.

FIG. 20 is a flow chart showing how the control unit of the preferred embodiment of the smart pillow system according to either FIG. 5 or FIG. 9 responds to changes in the position of the user of the smart pillow system when the head of the user is on the smart pillow system.

FIG. 21 is a perspective view of another preferred embodiment of a smart pillow system according to the disclosure showing shoulder pressure sensors in hidden line.

FIG. 22 is a plan view of the top of the smart pillow system of FIG. 21 showing the shoulder pressure sensors in hidden line.

FIG. 23 is a schematic showing the components and their interrelationship of the preferred embodiments of the smart pillow system of FIG. 21.

FIG. 24 is an illustrative X-Y diagram in which the pressure (P) detected by each of the shoulder pressure sensors is plotted with respect to the position (X) along the line of the shoulder pressure sensors when the user is lying on the smart pillow system of FIG. 21 while the user is in the supine position.

FIG. 25 is an illustrative X-Y diagram in which the pressure (P) detected by each of the shoulder pressure sensors is plotted with respect to the position (X) along the line of the shoulder pressure sensors when the user is lying on the smart pillow system of FIG. 21 while the user is in the side position.

FIG. 26 is a flow chart showing how the control unit of the preferred embodiments of the smart pillow system of FIG. 21 initializes with respect to the user.

FIG. 27 is a flow chart showing how the control unit of the preferred embodiment of the smart pillow system according to FIG. 21 responds to changes in the position of the user of the smart pillow system when the head and shoulders of the user is on the smart pillow system.

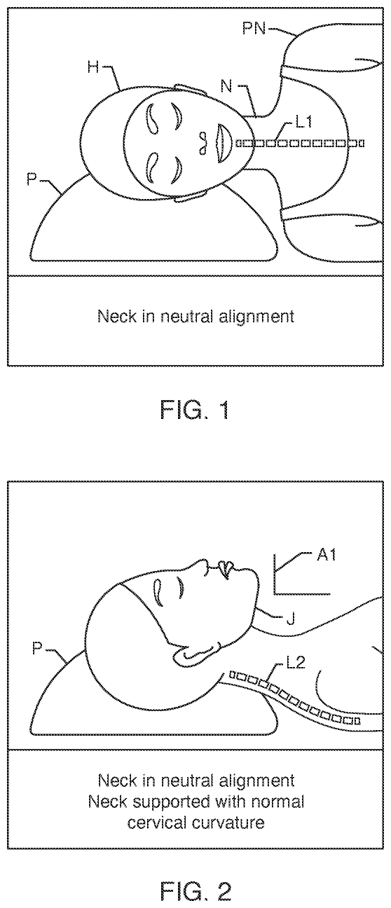

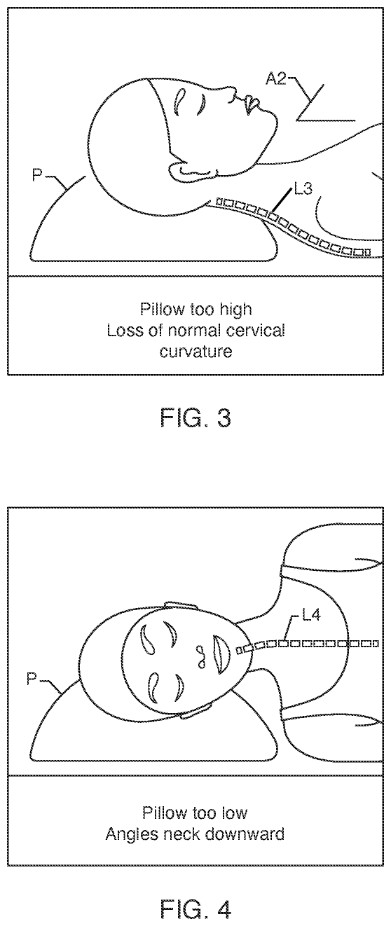

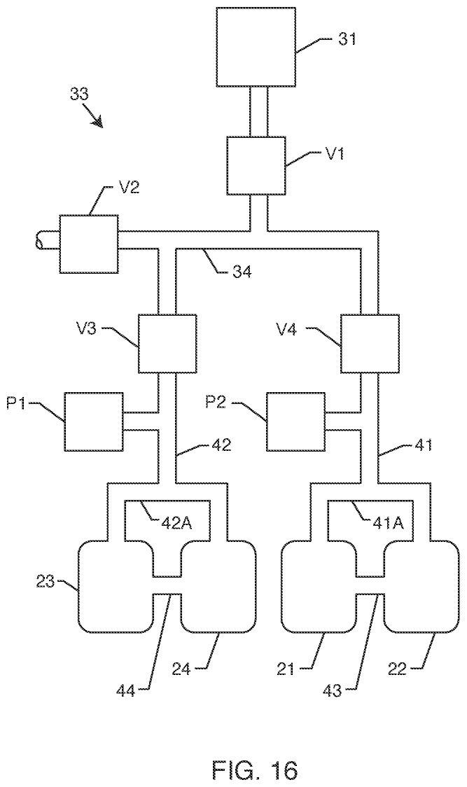

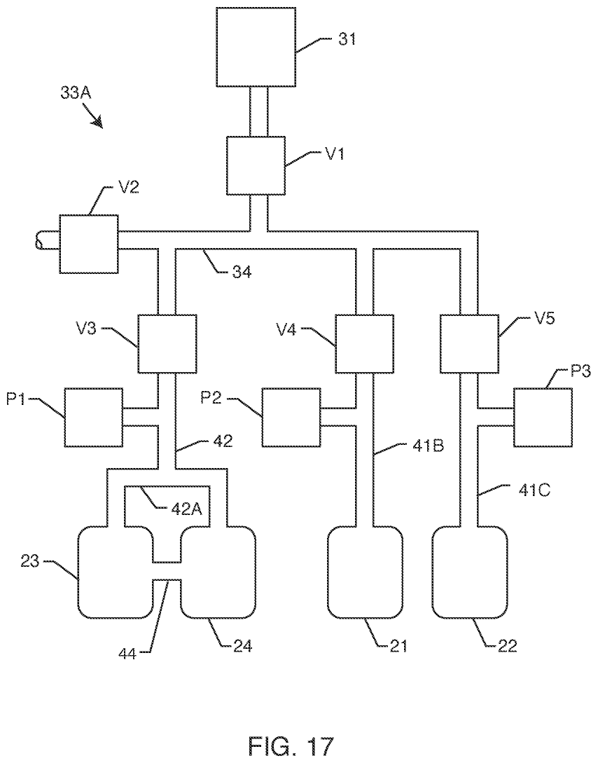

### REFERENCE NUMBER TABLE FOR THE DRAWINGS

| Reference No. | Description |
|---|---|
| P | pillow |
| PN | person |
| H | head |
| N | neck |
| L1 | interrupted line showing |
| | positioning of cervical |
| | spine in FIG. 1 |
| L2 | interrupted line showing |
| | positioning of cervical |
| | spine in FIG. 2 |
| L3 | interrupted line showing |
| | positioning of cervical |
| | spine in FIG. 3 |
| L4 | interrupted line showing |
| | positioning of cervical |
| | spine in FIG. 4 |
| J | jaw |
| A1 | angle of jaw line to |
| | horizontal, FIG. 2 |
| A2 | angle of jaw line to |
| | horizontal, FIG. 3 |
| 1 | smart pillow system, two |
| | air bag configuration |
| 1A | smart pillow system, four |
| | air bag configuration |
| 5 | top pillow exterior |
| 5A | rounded side edge |
| 5B | rounded top edges |
| 5C | cavity |
| 6 | higher top side |
| 7 | lower top side |
| 8 | speaker |
| 9 | cavity |
| 10 | heating pad |
| 11-1 | right bladder or air bag |
| 11-2 | left bladder or air bag |
| 11A | back of bladder 11-1 or 11- 2 |
| 11B | arm of bladder 11-1 or 11-2 |
| 11C | end of arm of bladder 11-1 or 11-2 |
| 11D | space defined between |
| | back 11A and arms 11B of |
| | bladder 1-1 or 11-2 |
| 12 | tray |
| 12A | front edge of tray 12 |
| 13 | securing ring |
| 14 | depression defined by |
| | bladder backs and arms |
| | (for configuration 1) |
| 20 | set of four bladders |
| 21 | left top bladder |
| 21A | back of left top bladder |
| 21B | arm of left top bladder |
| 21C | end of arm of left top |
| | bladder |
| 21D | space defined by back and |
| | arms of left top bladder |
| | 21 |
| 22 | right top bladder |
| 22A | back of right top bladder |
| | 22 |
| 22B | arm of right top bladder |
| | 22 |
| 22C | end of arm of right top |
| | bladder 22 |
| 22D | space defined by back and |
| | arms of right top bladder |
| | 22 |
| 23 | left bottom bladder |
| 23A | back of left bottom |
| | bladder 23 |
| 23B | arm of left bottom bladder |
| | 23 |
| 23C | end of arm of left bottom |
| | bladder 23 |
| 23D | space defined by back and |
| | arms of left bottom |
| | bladder 23 |
| 24 | right bottom bladder |
| 24A | back of right bottom |
| | bladder 24 |
| 24B | arm of right bottom |
| | bladder 24 |
| 24C | end of arm of right bottom |
| | bladder 24 |
| 24D | space defined by back and |
| | arms of right bottom |
| | bladder 24 |
| 24C | end of arm of right bottom |
| | bladder |
| 24D | space defined by back and |
| | arms of right bottom |
| | bladder 24 |
| 25 | depression defined by |
| | spaced bladders (for |
| | configuration 1A) |
| 26 | common webbing |
| | between adjacent bladders |
| 30 | bladder inflation system |
| 31 | air pump |
| 32 | control system unit (a/k/a |
| | controller) |
| 33 | pressure sensor and valve |
| | assembly corresponding |
| | to FIG. 16 |
| 33A | pressure sensor and valve |
| | assembly corresponding |
| | to FIG. 17 |
| 34 | manifold |
| 41 | tube going to bladders 21 |
| | and 22 |
| 41A | manifold tube (FIG. 16) |
| 41B | tube running between valve |
| | V4 and the bladder 21 (FIG. |
| | 17) |
| 41C | tube running between the |
| | valve V5 and the bladder 22 |
| | (FIG. 17) |
| 42 | tube going to bladders 23 |
| | and 24 (FIG. 16) |
| 42A | manifold tube (FIG. 16) |
| 43 | equalization tube joining |
| | bladders 21 and 22 (FIGS. 16) |
| 44 | equalization tube joining |
| | bladders 23 and 24 (FIGS. 16 |
| | and 17) |
| V1 | valve (FIGS. 16 and 17) |
| V2 | valve (FIGS. 16 and 17) |
| V3 | valve (FIGS. 16 and 17) |
| V4 | valve (FIGS. 16 and 17) |
| V5 | valve (FIG. 17) |
| P1 | pressure sensor (FIGS. 16 |
| | and 17) |
| P2 | pressure sensor (FIGS. 16 |
| | and 17) |
| P3 | pressure sensor (FIG. 17) |
| 100 | microprocessor |
| 102 | power rectifier voltage unit |
| 104 | pneumatic unit |
| 106 | air bags adjustment control |
| | unit |
| 107 | air bags |
| 108 | air pressure detection unit |
| 110 | air exhaust unit |
| 112 | Bluetooth ® transmission |
| | unit |
| 114 | mobile APP (installed on |
| | smart phone) |
| 116 | temperature control unit |
| 118 | music output unit |
| 120 | sound detection unit |
| 200 | start step |
| 202 | detect presence of subject |
| | (a/k/a user) (air bags are |
| | inflated to reference |
| | pressure) |
| 204 | subject lies on back or side |
| | step |
| 206 | detect and record pressure |
| | PX1 in air bag step |
| 208 | subject lies on side or back |
| | step |
| 210 | detect and record pressure |
| | PX2 in air bag step |
| 212 | compare magnitude of |
| | pressure PX1 to magnitude |
| | of pressure PX2 step |
| 214 | record pressure PX1 as |
| | supine position pressure |
| | and pressure PX2 as side |
| | position pressure if PX1 > |
| | PX2 step |
| 216 | record pressure PX1 as side |
| | position pressure and |
| | pressure PX2 as supine |
| | position pressure if PX1 < |
| | PX2 step |
| 218 | determine PX supine and |
| | PX side for inflation of air |
| | bags step (by averaging |
| | reference pressure with |
| | appropriate ones of PX1 |
| | and PX2) step |
| 220 | end step |
| 230 | start step |
| 232 | determine presence of |
| | sleeper (a/k/a user) step (air |
| | bags start at reference |
| | pressure) |
| 234 | detect head position of |
| | sleeper (by comparison to |
| | recorded PX1 and PX2 |
| | associated with head |
| | positions) step |
| 236 | determine whether air bags |
| | are at proper height for the |
| | detected head position of |
| | the sleeper (by comparing |
| | pressure in air bags to |
| | calculated pressure for that |
| | head position) step |
| 238 | adjust the air bags to proper |
| | height by inflating or |
| | deflating the air bags to |
| | calculated pressure for the |
| | head position step |
| 300 | smart pillow system with |
| | shoulder pressure detection |
| | system |
| 302 | main part of smart pillow |
| | system with shoulder |
| | pressure detection system |
| | (supports head) |
| 304 | extension of smart pillow |
| | system 300 between main |
| | part 302 and the bottom |
| | edge 306 |
| 306 | bottom edge of smart pillow |
| | system 300 |
| 310 | shoulder position detection |
| | system |
| 312 | strip containing shoulder |
| | pressure sensors |
| 314 | shoulder pressure sensors |
| | (individually labeled PS1- |
| | PS7) |
| 320 | start |
| 322 | detect presence of subject |
| | (a/k/a user) (air bags are |
| | inflated to reference |
| | pressure) |
| 324 | subject lies on back step |
| 326 | detect and record shoulder |
| | pressure profile as supine |
| | shoulder pressure profile |
| 328 | subject lies on side |
| 330 | detect and record shoulder |
| | pressure profile as side |
| | shoulder pressure profile |
| 332 | end |
| 350 | start |
| 352 | determine presence of |
| | sleeper (a/k/a user) step (air |
| | bags start at reference |
| | pressure) |
| 354 | detect shoulder position of |
| | sleeper (by comparison to |
| | recorded shoulder pressure |
| | profiles) and thus position |
| | of sleeper's body |
| 356 | determine whether air bags |
| | are at proper height for the |
| | detected position of the |
| | sleeper (by comparing |
| | pressure in air bags to |
| | calculated pressure for that |
| | position) |
| 358 | adjust the air bags to proper |
| | height by inflating or |
| | deflating the air bags to |
| | calculated pressure for the |
| | body position |

### DETAILED DESCRIPTION OF THE INVENTION

### 1. Physiology

The head of a human being is supported by the bones of the neck in a neutral alignment in which the cervical vertebrae of the neck have a natural curvature along a plane defined by the spine and extending through the front and rear of the body of the person. In neutral alignment, barring unusual conditions such as scoliosis, the cervical vertebrae have no curvature along a plane normal to the plane defined by the spine and extending through the front and rear of the body of the person. In other words, the cervical vertebrae in the neutral alignment curve forward from the shoulders but not side to side. The line of the jaw of the person's head when the neck is in neutral alignment is generally horizontal when the person is sitting or standing up and not appreciably pitched or angled up or down with respect to the horizontal.

When lying on a flat surface, as on a mattress or a floor, a person will not be able to achieve a neutral alignment of his or her neck without some support. Without support, the person's head is angled toward the flat surface when the person is lying on his or her back or side. The person's neck will not be in the neutral alignment. This position is uncomfortable and makes sleeping more difficult. Accordingly, a support under the head will help the person keep his or her neck in the neutral alignment when lying down. The support can be the person PN's own arms but more usually is a pillow.

FIG. 1 shows how a pillow P helps the person PN achieve a neutral alignment of her neck N when she is lying on her right side on a bed (the bed is not shown in the drawing). The pillow P supports the person PN's head H so that her neck N is not bent to the left or the right side of the person PN. The interrupted line L1 indicates the line of the vertebrae in her neck N as seen from her front or anterior side. The interrupted line L1 is straight as shown in FIG. 1, indicating that the person PN's neck N is aligned properly with respect to the lateral and medial directions. The pillow P may be used to achieve the same result when the person is lying on her left side on a bed.

FIG. 2 shows how a pillow P helps the person PN achieve a neutral alignment of her neck N when she is lying on her back on a bed (the bed is not shown in the drawing) and facing up. The pillow P raises her head H just enough to achieve a neutral alignment of her neck N. A neutral alignment of the neck N, as mentioned in paragraph 100531 above, involves a curve of the neck N towards and then away from the person PN's anterior or front side, as indicated by the curved interrupted line L2. The right angle A1 indicates that the line of the jaw J is generally vertical and not pitched or angled away from her chest or toward her chest. This angle A1 indicates that the person PN's neck N is in neutral alignment.

FIG. 3 shows how a pillow P that is too high or thick prevents the person PN from achieving neutral alignment of her neck N when she is lying on her back. The pillow P raises her head H so that the head H is tilted toward her chest. This prevents a neutral alignment of the person's neck N. The acute angle A2 indicates that the line of the jaw J is generally not vertical and in fact is pitched or angled toward the person N's chest. The curve of the neck N towards the person PN's anterior or front side, as indicated by the interrupted line L3, is too great. The person PN's neck N is out of neutral alignment with respect to the anterior and posterior directions.

Although not shown in the drawings, a pillow P that is too thin (or non-existent) also may prevent the person PN from achieving neutral alignment of her neck N when she is lying on her back, because the neck N has less of its normal curvature. This will tend to cause discomfort.

FIG. 4 shows how a pillow P that is too thin prevents the person PN from achieving neutral alignment of her neck N when she is lying on her right side. The pillow P supports the person PN's head H too close to the mattress of the bed (the mattress is not shown in the drawing) so that the person PN's neck N is bent down and out of neutral alignment. The cervical spine of the neck N is bent towards the person PN's right side, as indicated by the interrupted line L4, and is not straight, as it should be when the neck N is in neutral alignment with respect to the lateral and medial directions. The pillow P will tend to have the same result when the person is lying on her left side.

Although not shown in the drawings, a too-thick pillow P may prevent the person PN from achieving neutral alignment of her neck N when she is lying on her side on the mattress of a bed by forcing the neck N to curve upwardly and thus not achieving a neutral alignment.

A traditional pillow is usually comprised of a bag or flexible container with some sort of stuffing or material that deforms under the weight of a person's head to an extent. Traditional stuffing materials are feathers or down, buckwheat hulls, or their artificial equivalents such as polyester fiberfill and memory foam. The stuffing may not require a bag to contain the stuffing, such as one-piece foam pillows, but a covering is usually desirable for hygiene.

A person using a traditional pillow may deform the pillow, or add or subtract pillows, to provide sufficient support to the head in order to achieve a neutral alignment of the person's neck as described above. This preparation of traditional pillows requires some skill and also some readjustment when the person changes position. The selection of the pillows also is important to achieving the correct amount of support.

### 2. Preferred Embodiments of a Smart Pillow System

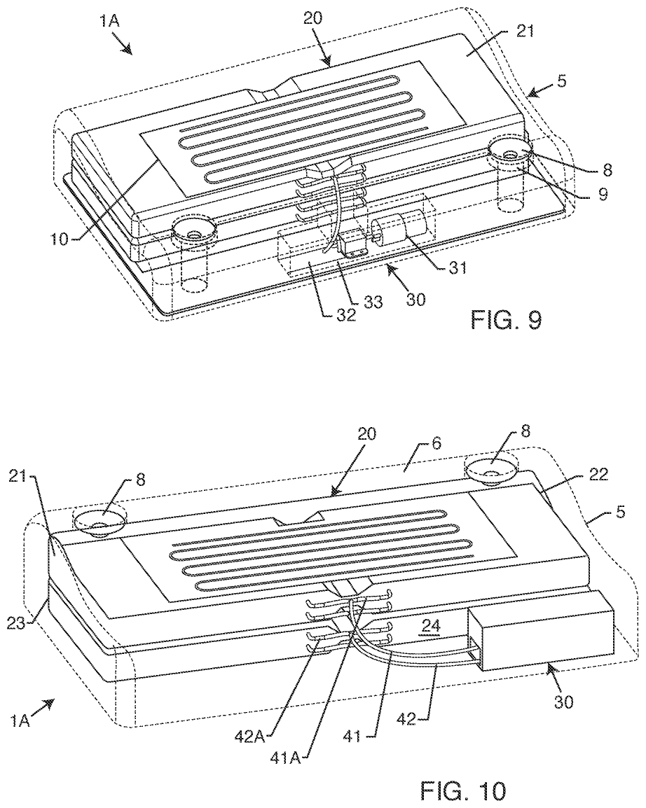

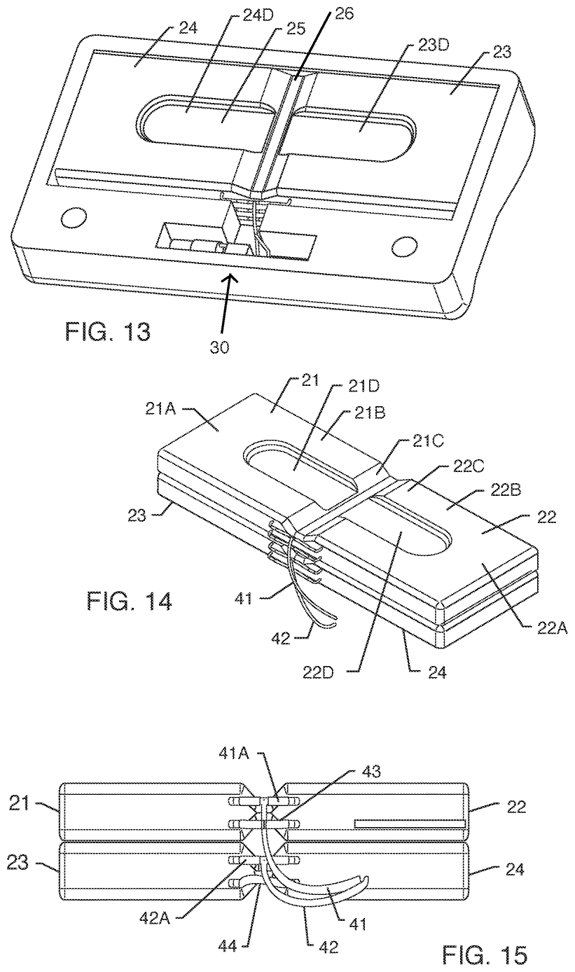

A preferred embodiment of a smart pillow system according to the disclosure varies the amount and location of support under a person's head in order to maintain the neutral alignment of the person's neck whether the person is lying on her back or on one side. FIGS. 5-8 depict the smart pillow system in a configuration 1 with two bladders or air bags. FIGS. 9-15 depict the smart pillow system in a configuration 1A with four bladders or air bags.

FIG. 5 shows the exterior of the smart pillow system of configuration 1. The smart pillow system 1 has a top pillow exterior 5 made of elastic and resilient foam, such as memory foam, that is shaped like a hollow parallelepiped box with rounded side edges 5A and rounded top edges 5B. A higher top side 6 of the top pillow exterior 5 is raised above the lower top side 7. A person using the smart pillow system 1 is intended to place her head H on the lower top side 7 with her neck N resting on the top side 6. Her head H should be between the speakers 8 resting in the cavities 9 formed in the top pillow exterior 5.

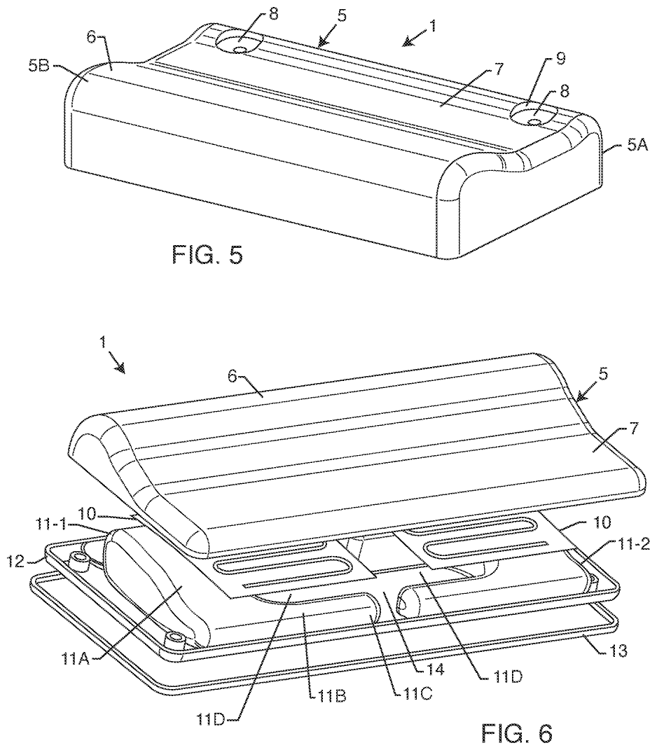

FIG. 6 shows some of the major components of the smart pillow system 1. (For the sake of simplicity, the speakers 8 are not shown in FIG. 6.) The top pillow exterior 5 defines a cavity 5C in order to accommodate the heating pads 10 and the bladders or air bags 11-1 and 11-2 resting on a tray 12 with the ring 13 available to secure the tray 12 to the top pillow exterior 5.

The left bladder 11-1 and the right bladder 11-2 shown in FIGS. 6-8 are similar to each other. Each has a shape like that of a rectangular horseshoe with a back 11A and two arms 11B, each arm having an end 11C. The bladders 11-1 and 11-2 are positioned with respect to each other so that the ends 11C of each of the bladders 11-1 and 11-2 are adjacent to the ends 11C of the other of the bladders 11-1 and 11-2 with the backs 11A of the bladders 11-1 and 11-2 being spaced from each other at opposite ends of the cavity 5C in the top pillow exterior. The spaces 11D between the back 11A and the arms 11B of the bladders 11-1 and 11-2 are empty and together with any space provided between the left bladder 11-1 and the right bladder 11-2 will define a depression 14.

The bladders 11-1 and 11-2 are made of a material that is generally inelastic, such as a plastic, and preferably are filled with air. The bladders 11-1 and 11-2 are sealed other than to admit or discharge air through an orifice and should not otherwise leak air. The bladders 11-1 and 11-2 will be filled with air at different pressures as needed to increase or decrease the height or thickness of the bladders 11-1 and 11-2 and thereby raise or lower the higher top side 6 and the lower top side 7 of the top pillow exterior 5 in order to position the head of a sleeping person as needed to maintain the neutral alignment of the person's cervical spine. The depression 14 formed by and between the bladders 11 will tend to cup the head of the person because the bladders 11-1 and 11-2 will not be pushing or supporting the top pillow exterior 5 in that region.

The two bladders 11-1 and 11-2 could be made as a single unit whereby their ends are joined. A unitary bladder would appear to be rectangular in outline and with a hole or depression in the middle. Instead of a hole, the unitary bladder could simply have crossbars between the sides of the bladder where the person's head is to rest in order to restrict the distance the bladder could expand in that region. A unitary bladder is simpler to operate because it requires fewer valves and pressure sensors, but is less flexible. It could not be made to rock the head of a person from side to side as discussed below in paragraph [00].

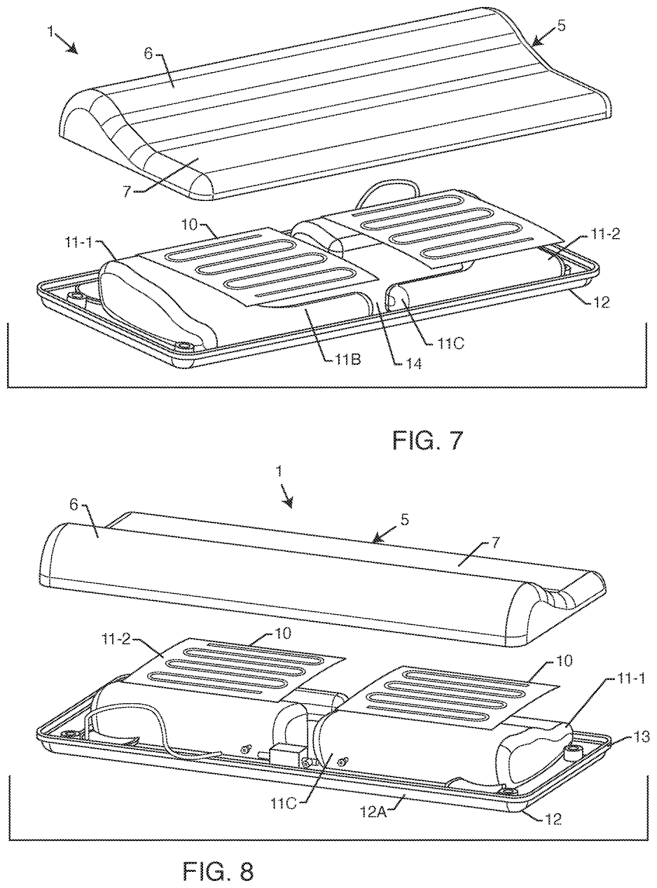

FIG. 7 shows the ring 13 in place on the tray 12 and thus positioned to attach the tray 12 to the top pillow exterior 5.

FIG. 8 shows the smart pillow system 1 rotated to show the top side 6 of the top pillow exterior 5 closer to the point of view. Some components to be discussed later, such as the air pump 31, are shown in the tray 12 between the bladders 11-1 and 11-2 and the front edge 12A of the tray 12. These components are provided to inflate or deflate the bladders 11-1 and 11-2.

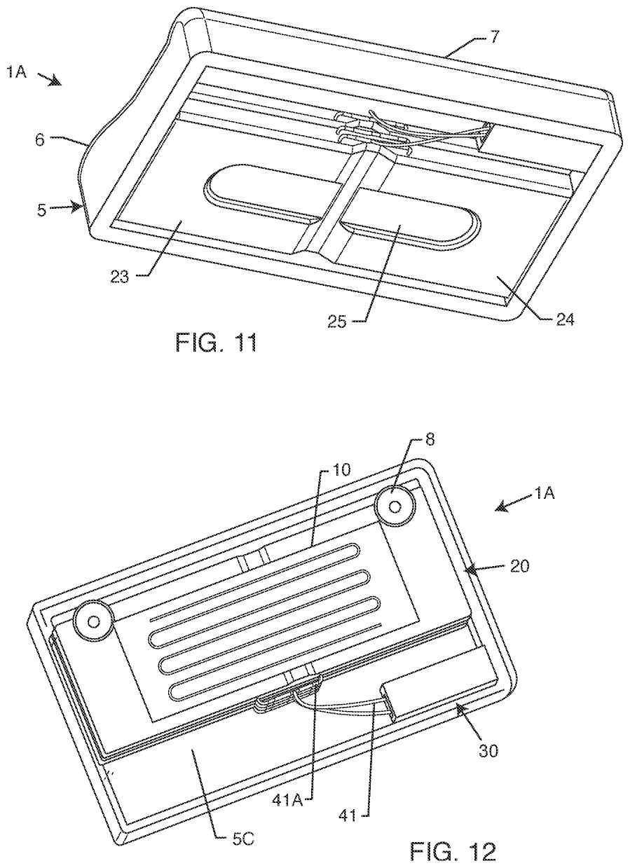

FIGS. 9-13 show another preferred embodiment of the smart pillow system, designated 1A. This embodiment 1A is like the embodiment of the smart pillow system 1 but in which the bladders 11-1 and 11-2 are replaced by a set 20 of four bladders 21, 22, 23, and 24. Each of these bladders is similar to the others in that it has the rectangular horseshoe shape or D-shape, as shown in FIGS. 11 and 13-15. For example, and referring to FIG. 14, the bladder 21 has a back 21A, two arms 21B each having an end 21C, and a space 21D defined between the back 21A and the arms 21C.

The bladder 21 is paired with the bladder 22 with the ends 21C of the bladder 21 adjacent the ends 22C of the arms 22B of the bladder 22. The back 21A of the bladder 21 is spaced from the back 22A of the bladder 22 so that the backs 21A and 22A are at opposite ends of the cavity 25 of the top pillow exterior 5. Preferably, the bladders 21 and 22 are joined to each other by a common webbing 26 that is part of the material from which the bladders 21 and 22 are formed, such as by stamping or gluing. This common webbing 26 will help maintain the position of the bladders 21 and 22 with respect to each other.

Underneath the pair of bladders 21 and 22 is the second pair of bladders 23 and 24. The second pair of bladders 23 and 24 preferably will have the same construction as the first pair of bladders 21 and 22. An advantage of providing overlapping pairs of bladders is that each pair of bladders does not have the greater range of heights that a single pair of bladders would have to have if only a single pair of bladders were to be provided.

FIGS. 9 and 10 are drawings which depict the top pillow exterior 5 as being transparent so one may see through it to underlying components, namely the set 20 of the four bladders 21-24 located within the cavity 5C defined in the top pillow exterior 5, the heating pad 10, the speakers 8 set in the cavities 9 formed in the top pillow exterior 5, and the bladder inflation system 30. In FIG. 9 the speakers 8 and their associated cavities 9 are located in the lower top side 7 of the top pillow exterior 5 whereas in FIG. 10 the speakers 8 and their associated cavities 9 are located in the raised top side 6 of the top pillow exterior 5 in order to show two possible locations for the speakers 8.

The bladder inflation system 30 is shown in FIGS. 9 and 10 as being located on the back side of the smart pillow system 1A and thus under the lower top side 7 of the top pillow exterior 5 (whereas in FIGS. 5-8 the bladder inflation system 30, which not shown in full in those drawings, is position on the front side of the smart pillow system 1 and thus under the raised top side 6 of the top pillow exterior 5). The bladder inflation system 30 comprises an air pump 31, a control system unit 32, and a pressure sensor and valve assembly 33. The bladder inflation system 30 supplies air to or removes air from the bladders (bladders 11-1 and 11-2 in the version of FIGS. 5-8 and the bladders 21-24 in the version of FIGS. 9-13) through tubing 40 in order to obtain the appropriate height of the bladders.

As shown in FIGS. 9-13, the tubes 41 and 42 join the bladder inflation control system 30 to the bladders 21-24. The tube 41 connects to the upper pair of bladders 21 and 22 by way of a manifold tube 41A and the tube 42 connects to the lower pair of bladders 23 and 24 by way of a manifold tube 42A. In addition, as shown in FIGS. 9, 10, 14, and 15, the upper pair of bladders 21 and 22 have an equalization tube 43 joining them and the lower pair of bladders 23 and 24 have a have an equalization tube 44 joining them. The equalization tubes 43 and 44 ensure that the bladders to which they are attached have the same pressure and thus height, if that is desired. However, the bladders of a pair of bladders, such as bladder 21 and 22, will have different pressures and thus heights when the smart pillow system 1A is to have the ability to rock the head of the user and thus the equalization tubes will need to have on-off valves controlled by the bladder inflation control system 30 or be omitted entirely, as discussed below in paragraph [0085]-[0086].

As will be discussed below in connection with FIG. 17, the upper pair of bladders 21 and 22 may be provided with air at different pressures in order to alternately raise and lower the height of those bladders, thereby gently rocking the head of the person using the smart pillow system 1A when snoring is detected by the control system unit 32. The bladders 21 and 22 will be provided with air through individually assigned tubes from the bladder inflation system 30 and no equalization tube 43 will join the bladders 21 and 22.

FIG. 16 is a schematic of the pressure sensor and valve assembly 33 and the air pump 31. The air pump 31 may operate all the time when the smart pillow system is activated or operate only when activated by the control system unit 32. In either case, a system of valves and pressure sensor inflates or deflates the bladders 21-24 as required to change their respective heights.

The first valve, V1, in line from the air pump 31 is closed to prevent air from passing through it from or to the pump 31. In effect, it isolates the pump 31 from the rest of the pressure sensor and valve assembly 33 but when open allows the air from the pump 31 to pass through valve V1.

A manifold 34 connects the valve V1 to the valves V2, V3, and V4. The purpose of the valve V2 is to permit air to pass out of the manifold 34 when the valve V2 is open and to prevent air to pass out of the manifold 34 when the valve V2 is closed. The valve V3, when open, permits air from the manifold 34 to either enter or exit the bladders 23 and 24 depending on the difference in pressure between the bladders 23 and 24 and the manifold 34. When closed, the valve V3 isolates the bladders 23 and 24 from the manifold 34. The valve V4, when open, permits air from the manifold 34 to either enter or exit the bladders 21 and 22 depending on the difference in pressure between the bladders 21 and 22 with respect to the manifold 34. When closed, the valve V4 isolates the bladders 21 and 22 from the manifold 34.

The bladders 23 and 24 are provided with a pressure sensor P1 downstream from the valve V3 that measures the air pressure in the bladders 23 and 24. The bladders 21 and 22 are provided with a pressure sensor P2 downstream from the valve V4 that measures the air pressure in the bladders 21 and 22. The pressure sensors P1 and P2 report their respective measured pressures to the control system unit 32.

Assuming that the pressure in the bladders 21 and 22 is too low to achieve the correct height of those bladders for achieving the desired neutral alignment of the cervical spine of the person using the smart pillow system 1A, the control system unit 32 will direct the air pump 31 to operate and open the valves V1 and V4 while closing (or keeping closed) the valves V2 and V3. This will permit air under pressure to enter the bladders 21 and 22 until the pressure sensor P2 senses the desired pressure. At that time the control system unit 32 will close the valve V4. Valve V1 preferably is closed in order to avoid over-pressurizing the manifold 34. The air pump 31 may be switched off or may continue running.

Now assuming that the pressure in the bladders 21 and 22 is too high to achieve the correct height of those bladders for achieving the desired neutral alignment of the cervical spine of the person using the smart pillow system 1A, the control system unit 32 will open the valves V2 and V4 while closing (or keeping closed) the valves V1 and V3. This will permit air to leave the bladders 21 and 22 and into the manifold 34 and then into the atmosphere through the valve V2 until the pressure sensor P1 senses the desired pressure. At that time the control system unit 32 will close the valve V2 and V4. Valve V1 preferably is closed in order to avoid over-pressurizing the manifold 34. The air pump 31 may be switched off or may continue running, as long as the valve V1 is closed.

The pressure in the bladders 23 and 24 is detected and altered the same way as described for the bladders 21 and 22, but using the pressure sensor P1 and the valve V3.

FIG. 17 is a schematic of a variation 33A of the pressure sensor and valve assembly 33 when the bladders 21 and 22 have the capability of having different heights and thus internal pressures. As in the configuration of FIG. 16, the air pump 31 may operate all the time when the smart pillow system 1A is activated or operate only when activated by the control system unit 32. In either case, a system of valves and pressure sensor inflates or deflates the bladders 21-24 as required to change their respective heights.

FIG. 17 shows how each of the bladders 21 and 22 has its own pressure supply and relief line and pressure sensor. The bladder 21 is connected to the manifold 34 through the valve V4 and the tube 41B. The bladder 22 is connected to the manifold 34 through the valve V5 and the tube 41C. The bladder 21 is connected to the pressure sensor P2 and the bladder 22 is connected to the pressure sensor P3. In effect, the manifold 34 is connected to three separate bladder systems. By supplying air under pressure to or releasing air from the bladder 21 independently of the bladder 22, and vice versa, the head of the person using the smart pillow system 1A may be rocked gently side to side as mentioned in paragraph [0053].

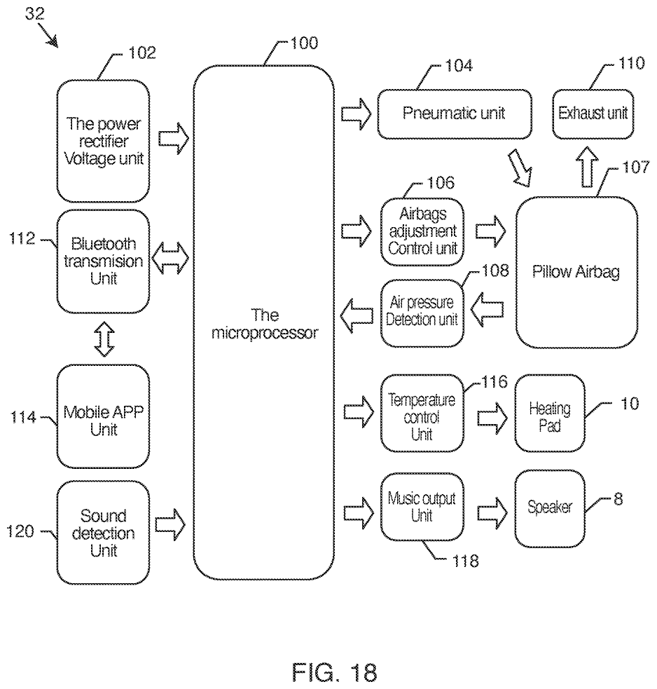

FIG. 18 is a schematic diagram of the control system unit 32 that is part of the bladder inflation system 30. At its heart is a microprocessor 100 that receives information about the pressure in the bladders or air bags, controls the pressure in each bladder by opening and controlling valves and the air pump according to memorized or prescribed values, interacts with a smart phone through a Bluetooth® short range wireless connection or the like, detects sound in order to rock the head of a snoring person or otherwise interrupt snoring, such as by playing music or other sounds, controls the temperature of the heating pad 10, and plays music through the speakers 8 to lull the user to sleep.

A power rectifier voltage unit 102 powers the control system unit 32 by providing electric power to the microprocessor 100, and components such as the various valves, the air pump 31, the speakers 8, and the heating pad 10. The source of power is the mains rectified by an adapter. An alternative power supply would be a battery that is recharged by cable or electromagnetic induction. That configuration would have the advantage of not requiring a cable to be connected to the smart pillow system 1 or 1A during use.

The pneumatic unit 104 comprises the air pump 31 and the airbags adjustment control unit 106 comprises the pressure sensor and valve assembly 33 or 33A discussed above in connection with FIGS. 16 and 17. As shown in FIG. 18, it also has an air pressure detection unit 108 and an exhaust unit 110. The air pressure detection unit 108 measures the ambient air pressure in order to adjust the pressure in the bladders (the bladders are indicated by reference number 107 in FIG. 18) will be necessary to achieve the desired height or thickness of the bladders. At higher altitude or at lower atmospheric pressures the amount of air to be pumped into a bladder will be less to achieve a desired height or thickness of the bladder. The exhaust unit 110 corresponds to the valve V2 on the manifold 34 that permits air to escape from the manifold 34 as described in the discussion of FIGS. 16 and 17.

The Bluetooth® short distance wireless transmission unit 112 connects the microprocessor 100 to a smart phone with a program or app 114 that is designed for the smart pillow system 1 or 1A. The app can be used to set the heights of the bladders when a sleeper is on her back or side as described further below.

The temperature control unit 116 sets the temperature of the heating pad 10 as desired by the person using the embodiments of the smart pillow system 1 or 1A, using the app 114 on a smart phone connected by Bluetooth® short range wireless technology to the microprocessor 100. Alternatively, the temperature control unit 116 may be programed to set the temperature of the heating pad 10 in relation to the ambient temperature.

The music output unit 118 plays music through the speakers 8. The music may be preset or selected using the app 114 on a smart phone connected by Bluetooth® short range wireless technology to the microprocessor 100. The app 114 will also permit control of the volume of the music.

The sound detection unit 120 incorporates a microphone and is capable of detecting when the person using the smart pillow system is snoring using known techniques of sound analysis, such as that taught by WO 2021064467 A1 to Okamura for “apparatus and method for snoring sound detection based on sound analysis,” the disclosure of which is incorporated by reference, or currently available in commercial products such as the Bedokey Memory Foam Pillows, sold on Amazon.com, which “detects snoring sounds.” The app 114 may be used to select a response to the snoring ranging from no action, to playing music, to gently rocking the head of the person to induce cessation of the snoring. To rock the head, the smart pillow system 1 preferably should have the ability to rock the head by independent inflation and deflation of the bladders on either side of the head, as in the system of FIG. 17. Alternatively, the smart pillow system could inflate and deflate connected bladders as in the system of FIG. 16 in order to cause the head to rise and fall. However, such action will be likely to take the cervical spine out of neutral alignment, if only temporarily.

The microprocessor 100 controls the pressure sensor and valve assembly 33 to inflate or deflate the bladders or air bags to the correct height required to achieve a neutral alignment of the cervical spine of a person using the smart pillow system 1 or 1A whether the person is on her back (supine) or on her side. This function requires the microprocessor 100 to detect whether the person is on her back or side, calculate the amount of pressure in each bladder needed in view of the ambient pressure to achieve the desired height of the bladder, and then activate pump and valves while monitoring pressure.

When a person has her head on the smart pillow system 1 or 1A, the back of the head will contact the smart pillow system lor 1A when the person is on her back whereas the side of the head will contact the smart pillow system 1 or 1A when the person is on her side. Most people's heads are shaped so that the side of the head has a greater area than the back of the head. As a result the pressure per unit area of the smart pillow system 1 or 1A is greater when the back of the head is resting on the smart pillow system 1 or 1A than when the side of the head is resting on the smart pillow system 1 or 1A. The greater pressure exerted per unit area will result in a greater pressure measured by the pressure sensors in at least a top layer of bladders or airbags because a spread-out force of the head needs less pressure to sustain it.

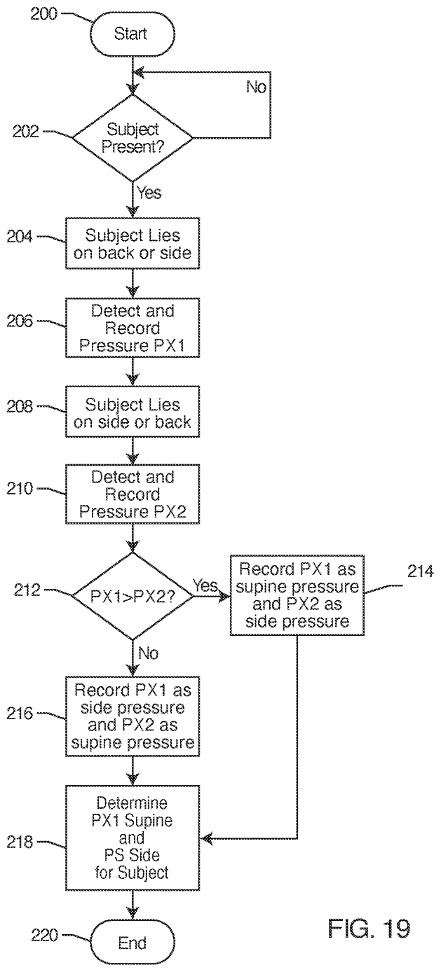

The difference in the pressures measured in the pressure sensors in at least the top layer of bladders will thus indicate the positioning of the head. This will be called the “delta pressure method” for determining the position of the head. A decision tree or flow chart for use of the delta pressure method of determining the position of the user's head is shown in FIG. 19. A change to a higher-pressure reading indicates the person has assumed the supine position and as the discussion concerning FIGS. 1-4 indicates, the height of the smart pillow system should be lower. A change to a lower pressure reading indicates that the person has changed to lying on her side whereby the smart pillow system should be higher for a side sleeper.

In step 200 the initialization process of the smart pillow system 1 or 1A starts by turning on the smart pillow system 1 or 1A. The smart pillow system 1 or 1A may be turned on using the app 114.

Step 202 requires the smart pillow system 1 or 1A to determine whether a person has her head on the smart pillow system 1 or 1A. Before the person puts her head on the pillow, the bladders are inflated to a predetermined pressure (the “reference pressure” or PX0). When the pressure increases from the reference pressure the microprocessor 100 will decide that the person has her head on the smart pillow system 1 or 1A.

In step 204 the person, following prompts from the app 114, lies on her back or side in order to measure the pressure of the air in the bladders for that position. For the sake of example, we will assume the person is on her back and the back of her head is resting on the smart pillow system 1 or 1A.

In step 206 the smart pillow system 1 or 1A records the air pressures PX1 it senses in the bladders and assigns the reading to the appropriate positioning of the head. In the example, this will be the pressures associated with the back of the head on the smart pillow system 1 or 1A.

In step 208 the person is directed to change position. In the example begun above, the person switches to a side sleeping position in which the side of her head rests on the smart pillow system 1 or 1A.

In step 210 the smart pillow system 1 or 1A records the air pressures PX2 it senses in the bladders and assigns the reading to the appropriate positioning of the head. In the example, these will be the pressures associated with the side of the head on the smart pillow system 1 or 1A.

In step 212 the pressures PX1 and PX2 are compared. If PX1 is greater than PX2, then in step 214 PX1 is assigned as the supine position pressure and PX2 as the side position pressure.

If, however, PX2 is greater than PX1, then in step 216 PX2 is assigned as the supine position pressure and PX1 as the side position pressure.

Preferably, the bottom two bladders 23 and 24 of the smart pillow system 1A are initially inflated to a given pressure and are not thereafter inflated. The pressures of the upper two bladders 21 and 22 of the smart pillow system 1A are the pressures to be measured and those bladders will have air added or released as needed to achieve the correct heights of the smart pillow system 1A for neutral cervical spinal alignment.

In step 218 the smart pillow systems 1 or 1A calculate the pressures to be set in the bladders 11-1 and 11-2 or 21 and 22 to achieve neutral alignment of the person's cervical spine for the positions of the person's head.

In step 220 the initialization process is ended.

Step 218, the calculation of the pressures to be set the bladders 11-1 and 11-2 or 21 and 22 to achieve neutral alignment of the person's cervical spine for the positions of the person's head, may be accomplished in one of a number of ways that will now be discussed.

The pressures to be induced in the bladders or air bags of the smart pillow system 1 or 1A to achieve correct pillow height can determined by either personalized or generalized methods. In the personalized method, the person would be asked to lie with her head on the smart pillow system 1 or 1A in one of the positions (e.g., supine with back of head contacting the smart pillow system 1 or 1A). A trained professional would observe the person and direct inflation or deflation of the bladders using the app 114 until the professional determines that the height of the smart pillow system is such that the person has achieved a neutral alignment of the cervical spine. The professional would use the app 114 to record the pressure in the bladders and connected with the supine position. Likewise, the person would move to a side sleeping position with the side of the head contacting the smart pillow system 1 or 1A and the professional would then carry out the same tasks of observation, inflation or deflation, and memorization using the app 114.

As part of the personalization process, the smart pillow system 1 or 1A would be trained to determine when the person is initially on her back or side by starting with a predetermined pressure in the bladders or air bags and measuring the pressures in those bladders when the person is on her back and on her side. When beginning use of the smart pillow system, for example at night, the microprocessor 100 will set the bladders of the smart pillow system 1 or 1A to the predetermined pressure and measure the pressure resulting from placing the person's head on the smart pillow system 1 or 1A to determine the starting position of the head.

The smart pillow system 1 or 1A would thereafter determine whether the person is on her back or side and activate the air pump 31 and the valves as needed to inflate or deflate the bladders to achieve the correct height of the smart pillow system 1 or 1A for that position.

The “personalized method,” as described above, offers the advantage of tailoring the smart pillow system 1 or 1A to provide the pressures and thus heights of the bladders for the positions of the person who will use it. However, it requires the participation of a trained professional who may not always be available. Accordingly, providing an initialization process that may be carried out by the ordinary user is desirable for a commercial smart pillow system. This will be called a “generalized method.”

In step 218 (see FIG. 19) the PX1 and PX2 pressures (and the associated positions of the person's head) could be compared to a table of pressures and positions that is created by observation of many individuals to determine the proper bladder inflation pressures to achieve the heights of the bladders needed to obtain neutral alignment of the cervical spine. The proper bladder inflation pressures are then associated with the supine position pressures and side position pressures for use thereafter. The user would input her height and weight using the app 114 and the microprocessor 100 would select the appropriate bladder inflation pressures for that person. This generalized method may be called the “survey method” for determining the proper inflation pressures.

A second generalized method, the “pressure averaging method,” may be used to determine the proper bladder inflation pressures to achieve the heights of the bladders needed to obtain neutral alignment of the cervical spine. The PX1 and PX2 pressures are individually averaged with the reference pressure PX0 to determine the pressures to be applied in the bladders 11-1 and 11-2 or bladders 21 and 22.

Thus, assuming that PX1 is the pressure in the bladders 11-1 and 11-2 or 21 and 22 associated with the supine position of the person with the back of the person's head on the smart pillow 1 or 1A, the pressure of the air in the bladders 11-1 and 11-2 or 21 and 22 should be set to the pressure (PX1+PX0)/2=PXsupine. Likewise, assuming that PX2 is the pressure in the bladders 11-1 and 11-2 or 21 and 22 associated with the side position of the person with the side of the person's head on the smart pillow 1 or 1A, the pressure of the air in the bladders 11-1 and 11-2 or 21 and 22 should be set to the pressure (PX2+PX0)/2=PXside.

Because PX1 is usually greater than PX2 when the positions of the person are so assigned, the calculated supine pressure PXsupine will be greater than the calculated side pressure PXside. The air bags 11-1 and 11-2 or 21 and 22 will be capable of achieving an appropriate height when the person is in the supine position, bearing in mind that when the head will be exerting a greater pressure per unit area than in the side position.

A third generalized method, the “shoulder width and weight/height method,” may be used to determine the proper bladder inflation pressures to achieve the heights of the bladders needed to obtain neutral alignment of the cervical spine. This method requires the user to input his or her measured shoulder width and weight to the smart pillow system 1 or 1A using the app 114.

A pre-calculated table stored in the microprocessor 100 relates the measured shoulder width to the height and thus the pressure of the bladders 11-1 and 11-2 (smart pillow system 1) or bladders 21 and 22 (smart pillow system 1A) when the user is in the side position. Wider shoulders will require more pressure in the bladders and thus more height of those pillows. The amount of pressure and thus height for the side position in relation to the user's shoulder width could be determined by observation of a number of users having different shoulder widths or by calculation on the basis of anatomy.

The weight/height of the user is related to the desired height of the pillows 11-1 and 11-2, or 21 and 22, for when the user is in the supine position. For an average person, meaning a person of a weight less than, for example, 250 pounds (113.4 kilograms) or a height of between, for example, five feet and six feet (1.52 to 1.83 meters), a pillow height of 1.5 inches (3.81 centimeters) will be satisfactory. (The smart pillow system 1A, having overlapping bladders, will have the pressures in those bladders set to have a combined height of 1.5 inches.)

The microprocessor 100 will assume the user is an average person as described above and the pressures of the bladders will be set for the height accordingly. Only if the user inputs values for weight or height outside the ranges would the microprocessor direct the use of different pressures and thus heights of the smart pillow system for the supine position. In this respect, the third generalized method is less costly than the first.

The smart pillow system is not limited to use by one person only. Another individual may use the smart pillow system, after that individual goes through the initialization process, in order for the smart pillow system 1 or 1A to assign bladder heights and thus pressures to that individual's head positions that will be assigned to that person. The app 114 may be used to identify the user to the microprocessor 100 in order for the control system 32 of the smart pillow system 1 or 1A to instruct the valves and air pump to inflate or deflate the bladders to achieve the proper heights for the positions of the user's head.

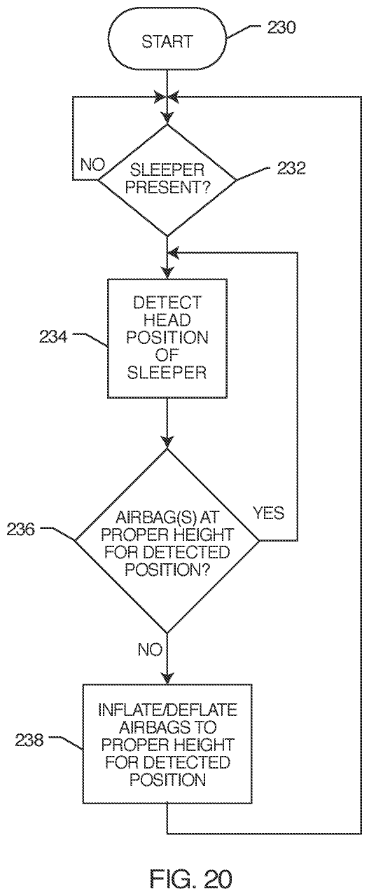

FIG. 20 is a simplified flow chart showing how the control system 32 of the smart pillow system 1 or 1A operates when a person (the “sleeper”) lies with her head on the smart pillow system 1 or 1A after performing the initialization process shown in FIG. 19 or, having previously performed that process, identifies herself to the smart pillow system 1 or 1A through the app 114.

The “start” step 230 refers to ensuring that the smart pillow system 1 or 1A is powered on. This can be done or checked through the app 114.

In step 232 the control system 32 determines whether pressure is being exerted on the bladders of the smart pillow system 1 or 1A. If so, a person is deemed to have her head on the smart pillow system 1 or 1A. If no pressure is exerted, than the control system 32 maintains readiness to determine whether pressure is being exerted.

If pressure is exerted and a person is deemed to have put her head on the smart pillow system 1 or 1A, then in step 234 the control system 32 determines from comparison to memorized values the position of the person's head.

In step 236 the control system 32 reviews the sensed pressures in the bladders or air bags to determine whether they are in the correct amounts to achieve the desired heights or thickness of the bladders for the position of the person's head. If yes, the control system 32 periodically goes back to step 234 to determine whether the position of the head of the person has changed.

If the sensed pressures in the bladders or air bags are too little or two great to achieve the desired heights or thickness of the bladders for the position of the person's head, then in step 238 the control system 32 inflates or deflates the bladders or air bags to achieve the desired heights of the bladders or air bags. The control system 32 then returns back to step 232. The user can stop or interrupt the process shown in FIG. 20 using the app 114 or, if no person is detected as being present for some predetermined time period, the control system 32 can shut itself down.

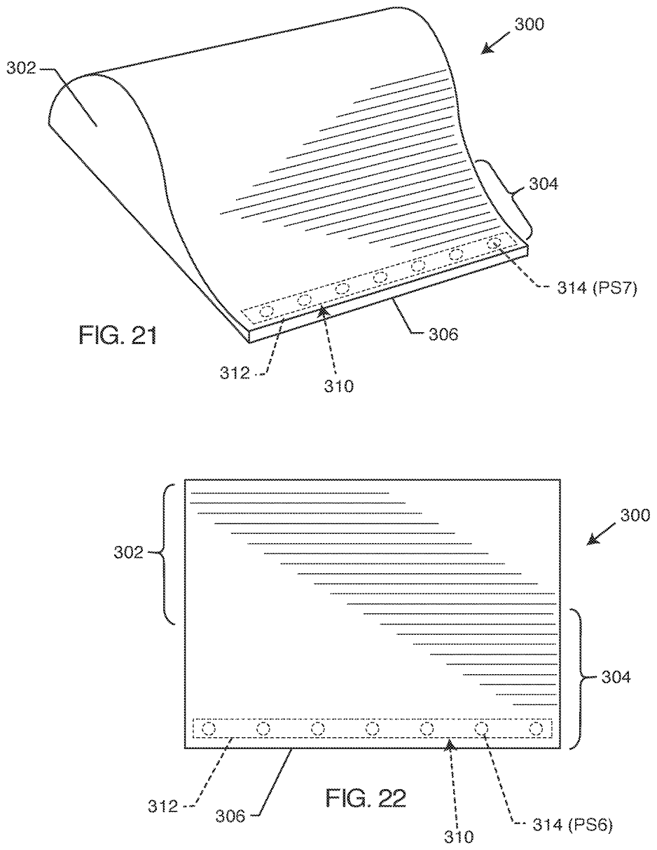

FIGS. 21 and 22 are perspective and top plan views, respectively, of another preferred embodiment of a smart pillow system according to the disclosure. This smart pillow system is given the reference number 300. The smart pillow system 300 internally will be similar to the embodiments of either FIGS. 5-8 and 9-15 but has a different system to detect the position (supine or side) of the user when the user is using the smart pillow system 300. This different system is the shoulder position detection system 310.

The shoulder position detection system 310 includes a strip 312 containing shoulder pressure sensors 314. The strip 312 is located in the extension 306 comprising the bottom edge 304 of the smart pillow system 300. The extension 306 is located between the main part 302 and the bottom edge 304 of the smart pillow system 300. The main part 302 contains the air bags and other components of the smart pillow system 300 and is intended to support the head of the user by expanding or contracting according to the position of the user as discussed above. The main part may have the shapes and components as described in connection with the smart pillow systems 1 and 1A.

The extension 306 narrows as it descends from the main part 302 to the bottom edge 304 of the smart pillow system 300 in order to accommodate the shoulders of the user without unduly raising those shoulders. The user will have his or her head resting on the main part 302 and one (side position) or both (supine position) of his or her shoulders resting on the extension 306.

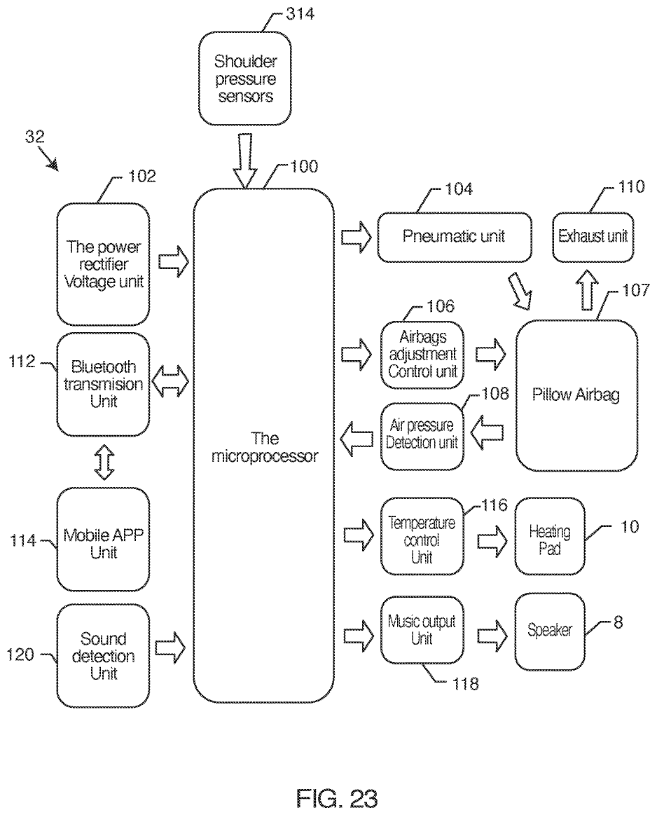

The strip 312 preferably is located close to the bottom edge 304 of the smart pillow system 300 and preferably is inside the extension 306. The strip 312 anchors the shoulder pressure sensors 314 in place with respect to each other and with respect to the smart pillow system 300 overall. The shoulder pressure sensors 314 detect the pressure exerted on them from above by the shoulders of the user and transmit their pressure readings by wire or Bluetooth® to the microprocessor 100 as shown in FIG. 23.

The structure of the smart pillow system 300, as shown in FIGS. 21 and 22, may have different configurations than that shown, as long as the shoulder pressure sensors 314 are in contact with the shoulder(s) of the user in order to sense the positioning (side or supine) of the body of the user. A smaller extension 306 or none at all is conceivable if the shoulder(s) touch, for example, the edge of the main part 302.

FIG. 23 shows the components of the smart pillow system embodiment 300. The components as shown are the same as those shown in FIG. 18 and are indicated by the reference numbers used in FIG. 18 and operate the same way except for how the position of the user is determined. FIG. 23 has the shoulder pressure sensors 314 that communicate with the microprocessor 100 in order to provide pressure readings that enable the microprocessor 100 to determine the position of the shoulders and thus the position of the user.

As shown in FIGS. 21 and 22, the strip 312 contains seven shoulder pressure sensors 314. More shoulder pressure sensors 314 may be provided along the strip 312 and will increase the reliability of the shoulder pressure readings. Providing too small a number of shoulder pressure sensors 314 may result in a loss of the ability to detect the positioning of the shoulders of the user.

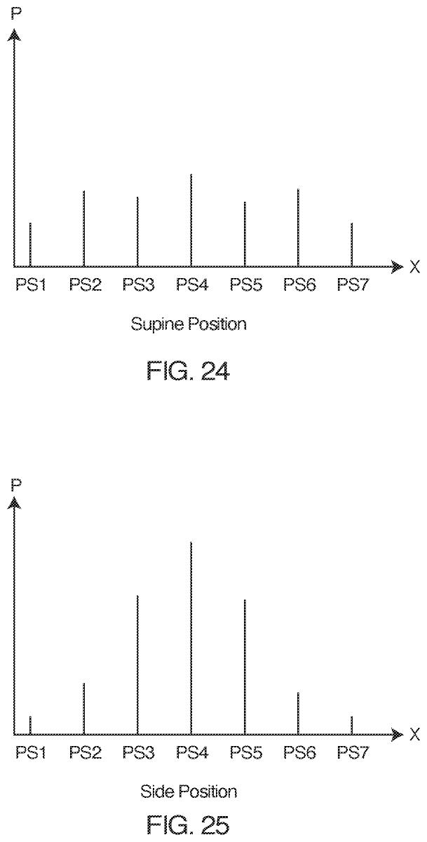

FIGS. 24 and 25 are diagrams that show examples of the pressure readings from the shoulder pressure sensors 314 along the strip 312 adjacent the bottom edge 304 of the smart pillow system 300. FIG. 24 shows example pressure readings PR1-PR7 from the seven shoulder pressure sensors 314, going from left to right as shown in FIG. 22, for the prone position. FIG. 25 shows example pressure readings PR1-PR7 from the seven shoulder pressure sensors 314 going from left to right as shown in FIG. 22, for the side position. The difference in the pressure readings for the two positions of the user is characteristic. A single and narrower pressure spike is indicative of the side position because a single shoulder is supporting the weight of the upper body whereas the supine position has both shoulders and the back to support the weight of the upper body.

The microprocessor 100 can be programmed to determine from the nature of the shoulder pressure readings whether the user is in the supine or side position. This is called the “shoulder pressure method.” This method requires an algorithm run by the microprocessor 100 that compares the shoulder pressure readings based on their variation or by comparison to a library of pressure readings to determine whether the user is resting on her back or one side.

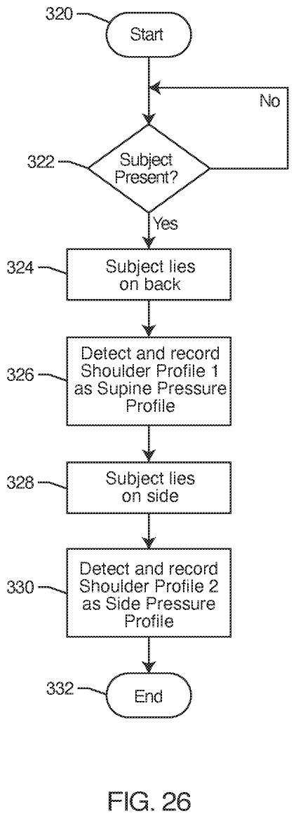

However, as explained above, providing an initialization process is desirable for a commercial smart pillow system. FIG. 26 is a flow chart showing the steps of such a process for the embodiment 300 of a smart pillow system.

In step 320 the initialization process of the smart pillow system 300 starts by turning on the smart pillow system 300. The user can turn on the smart pillow system 300 using the app 114.

Step 322 requires the smart pillow system 300 to determine whether a person has her head on the smart pillow system 300. Before the person puts her head on the pillow, the bladders are inflated to a predetermined pressure (the “reference pressure” or PX0) and when the pressure goes up the person is determined to have her head on the smart pillow system 300.

In step 324 the person, following prompts from the app 114, lies on her back or side in order to measure the pressures of the shoulder pressure sensors corresponding to that position. For the sake of example, we will assume the person is on her back, the back of her head is resting on the main part 302 of the smart pillow system 300, and her shoulders are situated over the strip 312 containing the shoulder pressure sensors 314.

In step 326 the smart pillow system 300 detects and records the pressure readings PS1-PS7 and assigns them to the appropriate positioning of the body of the user. In the example, this will be the pressure readings associated with both shoulders on the strip 312 when the user is in the supine position. The smart pillow system 300 will direct the adjustment of air pressures in the air bags to achieve the desired elevation of the head of the user for the supine position in order to achieve the neutral positioning of the spine of the user, as explained above.

In step 328 the user is directed to change position. In the example begun above, the user switches to a side sleeping position in which the side of her head rests on the main part 302 of the smart pillow system 300 and only one shoulder rests above the strip 312 containing the shoulder pressure sensors 314.

In step 330 the smart pillow system 300 detects and records the pressure readings PS1-PS7 and assigns them to the appropriate positioning of the body of the user. In the example, these will be the pressure readings associated with only one shoulder resting on the strip 312. The smart pillow system 300 will direct the adjustment of air pressures in the air bags to achieve the desired elevation of the head of the user for the side position in order to achieve the neutral positioning of the spine of the user, as explained above.

The initialization process ends in step 332.

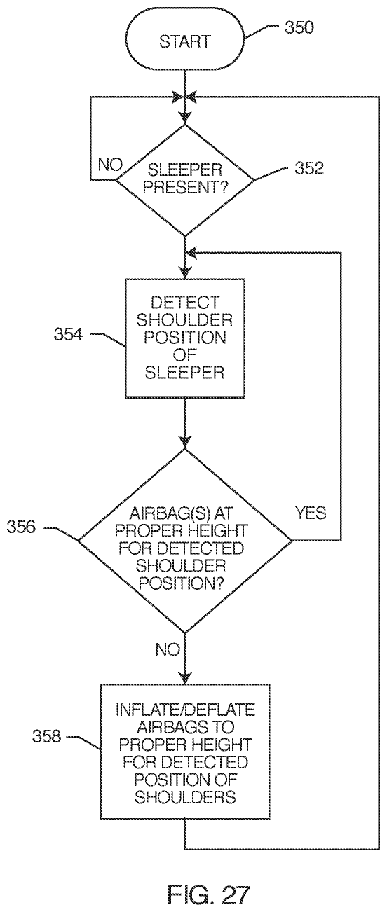

FIG. 27 is a simplified flow chart showing how the control system of the smart pillow system 300 operates when the user lies with her head on the main part 302 of the smart pillow system 300 after performing the initialization process shown in FIG. 26 or, having previously performed that process, identifies herself to the smart pillow system 300 through the app 114.

The “start” step 350 refers to ensuring that the smart pillow system 300 is powered on. The user can do this or check whether it has been done through the app 114.

In step 352 the control system determines whether pressure is being exerted on the bladders of the smart pillow system 300 and whether pressure is being exerted on the shoulder pressure sensors 314. If so, a person is deemed to have her head on the smart pillow system 300. If no pressure is exerted, than the control system 32 maintains readiness to determine whether pressure is being exerted.

If pressure is exerted and a person is deemed to have put her head on the main part 302 of the smart pillow system 300 and shoulders on the shoulder pressure sensors 314, then in step 354 the control system 32 determines, for example, from comparison to memorized values of shoulder pressures the position of the person's body (supine or side).

In step 356 the control system 32 reviews the sensed pressures in the bladders or air bags to determine whether they are in the correct amounts (determined by a personalized method or one of the generalized methods) to achieve the desired heights or thickness of the bladders for the position of the person's head. If yes, the control system 32 periodically goes back to step 354 to determine whether the position of the person has changed.

If the sensed pressures in the bladders or air bags are too little or too great to achieve the desired heights or thickness of the bladders for the position of the person's head, then in step 358 the control system 32 inflates or deflates the bladders or air bags to achieve the desired heights of the bladders or air bags corresponding to the detected position of the body of the user. The control system 32 then returns back to step 354. The user can stop or interrupt the process shown in FIG. 27 using the app 114 or, if no person is detected as being present for some predetermined time period, the control system 32 can shut itself down.

The invention is intended to cover alternatives, modifications and equivalents that may be included within the spirit and scope of the invention as defined by the appended claims.

What is claimed is:

1. A smart pillow system deformable by a controller for maintaining the cervical spine of a user who is a human being in a neutral alignment despite changes in the position of the user from lying on his or her side or on his or her back, comprising:
at least one inflatable bladder,
a controller, the controller comprising an air pump to increase air pressure in the at least one inflatable bladder, a pressure gauge to measure air pressure in the at least one inflatable bladder, and a valve to release air from the at least one inflatable bladder, and a microprocessor operatively connected to the air pump, pressure gauge, and valve to operate the same,
wherein the controller is adapted to measure the pressure of air in the first inflatable bladder and increases or decreases the pressure of the air in the at least one inflatable bladder in order to increase or decrease a height of the first inflatable bladder as needed to maintain the neutral alignment of the cervical spine of the user.

2. The smart pillow system according to claim 1 further comprising an encasement layer configured to enclose the at least one inflatable bladder.

3. The smart pillow system according to claim 1 wherein the controller is configured to calculate the pressure of the air in the at least one inflatable bladder to achieve the heights of the at least one inflatable bladder for the supine position and for the side position that will place the cervical spine of the user in a neutral alignment for the supine position and for the side position by using information from the user.

4. The smart pillow system according to claim 3 wherein the controller is configured to provide a measurement relating to a first pressure of the air in the at least one inflatable bladder when a user is lying with his or her head on the pillow with the at least one inflatable bladder underneath the head of the person and the person is in either a supine or a side position and a measurement relating to a second pressure of the air in the at least one inflatable bladder when the user is lying with his or her head on the pillow with the at least one inflatable bladder underneath the head of the person and the person is in the side or the supine position.

5. The smart pillow system according to claim 4 wherein the controller is configured to inflate the at least one inflatable bladder to a reference pressure prior to the user lying with his or her head on the pillow and prior to the controller providing the measurements of the first pressure of air and the second pressure of the air.

6. The smart pillow system according to claim 5 wherein the controller is configured to compare the measurements relating to the first and the second pressures and to assign the larger of the first and second pressures to the supine position and the smaller of the first and second pressures to the side position.

7. The smart pillow system according to claim 1 further comprising at least one shoulder pressure sensor adapted to register a pressure produced by a shoulder or shoulders of the user and communicate a pressure profile signal to the controller indicating the pressure produced by the shoulder or shoulders of the user wherein the controller is configured to determine from the pressure profile signal whether the user is in a supine or side position.

8. The smart pillow system according to claim 6 or 7 wherein the controller is configured to refer to a table of air pressures collected from observation of a plurality of persons to achieve the heights of the at least one inflatable bladder for the supine position and for the side position that will place the cervical spine of the user in a neutral alignment for the supine position and for the side position, the choice of which pressures being determined by information about the user's height and weight.

9. The smart pillow system according to claim 6 or 7 wherein the controller is configured to calculate the pressure of the air in the at least one inflatable bladder to achieve the heights of the at least one inflatable bladder for the supine position and for the side position that will place the cervical spine of the user in a neutral alignment for the supine position and for the side position by averaging the measured pressures of air associated with the respective positions with the reference pressure.

10. The smart pillow system according to claim 6 or 7 wherein the controller is configured to calculate the pressure of the air in the at least one inflatable bladder to achieve the heights of the at least one inflatable bladder for the side position by receiving information identifying the shoulder width of the user.

11. The smart pillow system according to claim 10 wherein the controller is configured to calculate the pressure of the air in the at least one inflatable bladder to achieve the heights of the at least one inflatable bladder for the supine position by receiving information identifying the height and/or weight of the user when such height and/or weight is outside a pre-determined range for which a standard pressure and thus height of the at least one bladder may be applied.

12. The smart pillow system according to claim 1 wherein the at least one inflatable bladder is configured to have a hollow area in the middle of the at least one inflatable bladder for receiving the head of the person.

13. The smart pillow system according to claim 1 further comprising a second inflatable bladder, wherein the at least one or first inflatable bladder and the second inflatable bladder together define a hollow area between them.

14. The smart pillow system according to claim 13 wherein and the controller is configured to separately inflate or deflate the first and second inflatable bladders.

15. The smart pillow system according to claim 13 wherein the first and second inflatable bladders are joined by a web.

16. The smart pillow system according to claim 13 wherein the first and second inflatable bladders are disposed, respectively, on right and left sides of the smart pillow system.

17. The smart pillow system according to claim 16 wherein the controller is provided with a microphone and is configured to detect the sound of snoring by the person and to inflate the first and second inflatable bladders unequally in order to rock the head of the person whereby the person will stop snoring.

18. The smart pillow system according to claim 13 further comprising a third inflatable bladder positioned beneath the first inflatable bladder and a fourth inflatable bladder beneath the second inflatable bladder wherein the controller is configured to inflate or deflate the third and fourth inflatable bladders in order to place the cervical spine of a human being in a neutral alignment for the supine position and for the side position.

19. The smart pillow system according to claim 1 further comprising a heating pad connected to the controller and wherein the controller sets the temperature of the heating pad.

20. The smart pillow system according to claim 1 further comprising a speaker connected to the controller wherein the controller is configured to play music or soothing sounds.

21. The smart pillow system according to claim 1 further comprising an app hosted by a smart phone for connecting with the controller by short range wireless technology communication, wherein the app identifies the user to the controller for application of the calculated pressures in the first inflatable bladder for the supine and side positions.

22. The smart pillow system according to claim 19 further comprising an app hosted by a smart phone for connecting with the controller by short range wireless technology communication, wherein the user may instruct the app to communicate to the controller to set the temperature of the heating pad to a desired temperature.

23. The smart pillow system according to claim 20 wherein the app hosted by a smart phone for connecting with the controller by a short range wireless technology communication, wherein the user may instruct the app to select from a library of music or soothing sounds to provide to the controller for playing through the speakers.

24. A method for operating a smart pillow system deformable by a controller for maintaining the cervical spine of a user who is a human being in a neutral alignment despite changes in the position of the user from lying on his or her side or on his or her back, comprising the following steps:
providing a smart pillow comprising at least one inflatable bladder, and a controller, the controller comprising an air pump to increase air pressure in the at least one inflatable bladder, a pressure gauge to measure air pressure in the at least one inflatable bladder, and a valve to release air from the at least one inflatable bladder, and a microprocessor operatively connected to the air pump, pressure gauge, and valve to operate the same,
inflating the at least one inflatable bladder to a reference pressure,
having the user lie with his or head on the smart pillow in one of the side or supine positions,
measuring and recording in the microprocessor the pressure of the air in the at least one inflatable bladder for the one of the side or supine positions,
having the user move to the other of the side or supine positions,
measuring and recording in the microprocessor the pressure of the air in the at least one inflatable bladder for the second position,
comparing in the microprocessor the recorded pressures of air to determine which is larger, and
assigning in the microprocessor the larger recorded pressure to the supine position and the smaller recorded pressure to the side position.

25. The method for operating a smart pillow system according to claim 24 further comprising the following steps:
calculating in the microprocessor the pressure to which the at least one inflatable pillow should be set for the supine position by averaging the larger recorded pressure with the reference pressure.

26. The method for operating a smart pillow system according to claim 25 further comprising the following steps:
calculating in the microprocessor the pressure to which the at least one inflatable pillow should be set for the side position of the user by averaging the smaller recorded pressure with the reference pressure.

27. A method for operating a smart pillow system deformable by a controller for maintaining the cervical spine of a user who is a human being in a neutral alignment despite changes in the position of the user from lying on his or her side or on his or her back, comprising the following steps:
providing a smart pillow comprising at least one inflatable bladder, and a controller, the controller comprising an air pump to increase air pressure in the at least one inflatable bladder, a pressure gauge to measure air pressure in the at least one inflatable bladder, and a valve to release air from the at least one inflatable bladder, a microprocessor operatively connected to the air pump, pressure gauge, and valve to operate the same, and one or more shoulder pressure sensors adapted to sense pressures exerted by the shoulders of the user and communicate these pressures to the microprocessor,
having the user lie with his or her head on the smart pillow system in one of the side or supine positions and his or her shoulders in contact with the smart pillow system wherein the shoulders will cause the shoulder pressure sensors to detect pressures exerted by the shoulders,
communicating the pressures sensed by the shoulder pressure sensors to the microprocessor,
analyzing in the microprocessor the pressures sensed by the shoulder pressure sensors to determine whether the user is in the side position or the supine position,
inflating the at least one bladder to a height determined for the sensed position of the user.
